(19) United States
(12) Patent Application Publication
(10) Pub. No.: US 2011/0041194 A1
(43) Pub. Date: Feb. 17, 2011

(54) I-MSOI HOMING ENDONUCLEASE VARIANTS HAVING NOVEL SUBSTRATE SPECIFICITY AND USE THEREOF

(75) Inventor: Sylvestre Grizot, La Garenne Colombes (FR)

Correspondence Address: KENYON & KENYON LLP, ONE BROADWAY, NEW YORK, NY, 10004, US

(73) Assignee: Cellectis, Romainville cedex (FR)

(21) Appl. No.: 12/745,261

(22) PCT Filed: Nov. 28, 2007
(86) PCT No.: PCT/IB07/04376

§ 371 Date: May 28, 2010

(51) Int. Cl. A01K 67/033; C12N 9/22; C07H 21/04; C12N 15/63; C12N 1/19; A01H 5/00; A61K 38/46; A61P 31/12; A01N 63/00; C12N 15/00; A01P 1/00

(52) U.S. Cl. 800/13; 435/199; 536/23.2; 435/320.1; 435/254.21; 800/298; 424/94.6; 435/440

(57) ABSTRACT

An I-MsoI homing endonuclease variant able to cleave mutant I-MsoI sites having variation at positions ±8 to ±10, a vector encoding said variant, a cell, an animal or a plant modified by said vector. Use of said I-MsoI endonuclease variant and derived products for genetic engineering, genome therapy and antiviral therapy.

The invention relates also to an I-MsoI homing endonuclease variant having novel substrate specificity, to a vector encoding said variant, to a cell, an animal or a plant modified by said vector and to the use of said I-MsoI endonuclease variant and derived products for genetic engineering, genome therapy and antiviral therapy.

Among the strategies to engineer a given genetic locus, the use of rare cutting DNA endonucleases such as meganucleases has emerged as a powerful tool to increase homologous gene targeting through the generation of a DNA double strand break (DSB). Meganucleases recognize large (>12 bp) sequences, and can therefore cleave their cognate site without affecting global genome integrity. Homing endonucleases, the natural meganucleases, constitute several large families of proteins encoded by mobile introns or inteins. Their target sequence is usually found in homologous alleles that lack the intron or intein, and cleavage initiates the transfer of the mobile element into the broken sequence by a mechanism of DSB-induced homologous recombination. I-SceI was the first homing endonuclease used to stimulate homologous recombination over 1000-fold at a genomic target in mammalian cells (Choulika et al., Mol. Cell. Biol., 1995, 15:1968-1973; Cohen-Tannoudji et al., Mol. Cell. Biol., 1998; 18:1444-1448; Donoho et al., Mol. Cell. Biol., 1998; 18:4070-4078; Alwin et al., Mol. Ther., 2005, 12:610-617; Porteus, M. H., Mol. Ther., 2006, 13:438-446; Rouet et al., Mol. Cell. Biol., 1994, 14:8096-8106). Recently, I-SceI was also used to stimulate targeted recombination in mouse liver in vivo, and recombination could be observed in up to 1% of hepatocytes (Gouble et al., J. Gene Med., 2006, 8:616-622). However an inherent limitation of such a methodology is that it requires the prior introduction of the natural cleavage site into the locus of interest since the repertoire of sequences cleavable by natural meganucleases is too limited to address the complexity of the genomes, and there is usually no cleavable site in a chosen gene. To circumvent this limitation, significant efforts have been made over the past years to generate endonucleases with tailored cleavage specificities. Such proteins could be used to cleave genuine chromosomal sequences and open new perspectives for genome engineering in wide range of applications. For example, meganucleases could be used to induce the correction of mutations linked with monogenic inherited diseases, and bypass the risk due to the randomly inserted transgenes used in current gene therapy approaches (Hacein-Bey-Abina et al., Science, 2003, 302, 415-419).

Fusion of Zinc-Finger Proteins (ZFPs) with the catalytic domain of the FokI, a class IIS restriction endonuclease, were used to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such nucleases could recently be used for the engineering of the ILR2G gene in human cells from the lymphoid lineage (Urnov et al., Nature, 2005, 435, 646-651).

The binding specificity of Cys2-His2 type Zinc-Finger Proteins, is easy to manipulate, probably because they represent a simple (specificity driven by essentially four residues per finger), and modular system (Pabo et al., Annu. Rev. Biochem., 2001, 70, 313-340; Jamieson et al., Nat. Rev. Drug Discov., 2003, 2, 361-368. Studies from the Pabo (Rebar, E. J. and C. O. Pabo, Science, 1994, 263, 671-673; Kim, J. S, and C. O. Pabo, Proc. Natl. Acad. Sci. USA, 1998, 95, 2812-2817), Klug (Choo, Y. and A. Klug, Proc. Natl. Acad. Sci. USA, 1994, 91, 11163-11167; Isalan M. and A. Klug, Nat. Biotechnol., 2001, 19, 656-660) and Barbas (Choo, Y. and A. Klug, Proc. Natl. Acad. Sci. USA, 1994, 91, 11163-11167; Isalan M. and A. Klug, Nat. Biotechnol., 2001, 19, 656-660) laboratories resulted in a large repertoire of novel artificial ZFPs, able to bind most G/ANNG/ANNG/ANN sequences.

Nevertheless, ZFPs might have their limitations, especially for applications requiring a very high level of specificity, such as therapeutic applications. The FokI nuclease activity in fusion acts as a dimer, but it was recently shown that it could cleave DNA when only one out of the two monomer was bound to DNA, or when the two monomers were bound to two distant DNA sequences (Catto et al., Nucleic Acids Res., 2006, 34, 1711-1720). Thus, specificity might be very degenerate, as illustrated by toxicity in mammalian cells (Porteus, M. H. and D. Baltimore, Science, 2003, 300, 763) and Drosophila (Bibikova et al., Genetics, 2002, 161, 1169-1175; Bibikova et al., Science, 2003, 300, 764-.).

Given their exquisite specificity, homing endonucleases may represent ideal scaffolds for engineering tailored endonucleases. Several studies have shown that the DNA binding domain from LAGLIDADG proteins, the most widespread homing endonucleases (Chevalier, B. S, and Stoddard B. L., Nucleic Acids Res. 2001; 29:3757-74) could be engineered. LAGLIDADG refers to the only sequence actually conserved throughout the family and is found in one or more often two copies in the protein (Lucas et al., Nucleic Acids Res., 2001, 29:960-969). Proteins with a single motif, such as I-CreI and I-MsoI, form homodimers and cleave palindromic or pseudo-palindromic DNA sequences, whereas the larger, double motif proteins, such as I-SceI are monomers and cleave non-palindromic targets. Several different LAGLIDADG proteins have been crystallized, and they exhibit a very striking conservation of the core structure that contrasts with the lack of similarity at the primary sequence level (Jurica et al., Mol. Cell., 1998; 2:469-476; Chevalier et al., Nat. Struct. Biol. 2001; 8:312-316; Chevalier et al., J. Mol. Biol., 2003, 329:253-69, Moure et al., J. Mol. Biol., 2003, 334:685-695; Moure et al., Nat. Struct. Biol., 2002, 9:764-770; Ichiyanagi et al., J. Mol. Biol., 2000, 300:889-901; Duan et al., Cell, 1997, 89:555-564; Bolduc et al., Genes Dev., 2003, 17:2875-2888; Silva et al., J. Mol. Biol., 1999, 286:1123-1136). In this core structure, two characteristic αββαββα folds, contributed by two monomers, or by two domains in double LAGLIDAG proteins, are facing each other with a two-fold symmetry. DNA binding depends on the four β strands from each domain, folded into an antiparallel β-sheet, and forming a saddle on the DNA helix major groove. The catalytic core is central, with a contribution of both symmetric monomers/domains. In addition to this core structure, other domains can be found: for example, PI-SceI, an intein, has a protein splicing domain, and an additional DNA-binding domain (Moure et al., Nat. Struct. Biol., 2002, 9:764-70, Grindl et al., Nucleic Acids Res. 1998, 26:1857-1862).

Several LAGLIDAG proteins, including PI-SceI (Gimble et al., J. Mol. Biol., 2003, 334:993-1008), I-CreI (Seligman et al., Nucleic Acids Res. 2002, 30:3870-3879; Sussman et al., J. Mol. Biol., 2004, 342:31-41; International PCT Applications WO 2006/097784, WO 2006/097853, WO 2007/060495 and WO 2007/049156; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J Am Chem. Soc., 2006, 128:2477-2484) and I-MsoI (Ashworth et al., Nature, 2006, 441:656-659) could be modified by rational or semi-retional mutagenesis and screening to acquire new binding or cleavage specificities.

Another strategy was the creation of new meganucleases by domain swapping between I-CreI and I-DmoI, leading to the generation of a meganuclease cleaving the hybrid sequence corresponding to the fusion of the two half parent target sequences (Epinat et al., Nucleic Acids Res., 2003, 31:2952-2962; Chevalier et al., Mol. Cell. 2002, 10:895-905; International PCT Applications WO 03/078619 and WO 2004/031346).

Recently, semi rational design assisted by high throughput screening methods allowed to derive thousands of novel proteins from I-CreI (Smith et al., Nucleic Acids Res. 2006, 34, e149; Arnould et al., J. Mol. Biol., 2006, 355:443-458; International PCT Applications WO 2006/097784, WO 2006/097853, WO 2007/060495 and WO 2007/049156). In such an approach, a limited set of protein residues are chosen after examination of protein/DNA cocrystal structure, and randomized. Coupled with high-throughput screening (HTS) techniques, this method can rapidly result in the identification of hundreds of homing endonucleases derivatives with modified specificities.

Furthermore, DNA-binding sub-domains that were independent enough to allow for a combinatorial assembly of mutations were identified (Smith et al., Nucleic Acids Res. 2006, 34, e 149; International PCT Applications WO 2007/049095 and WO 2007/057781). These findings allowed for the production of a second generation of engineered I-CreI derivatives, cleaving chosen targets. This combinatorial strategy, has been illustrated by the generation of meganucleases cleaving a natural DNA target sequence located within the human RAG1 and XPC genes (Smith et al., Nucleic Acids Res., 2006, 34, e149; Arnould et Mol. Biol., 2007, 371:49-65; International PCT Applications WO 2007/093836 and WO 2007/093918).

However, although the capacity to combine up to four sub-domains considerably increases the number of DNA sequences that can be targeted, it is still difficult to fully appreciate the range of sequences that can be reached. One of the most elusive factors is the impact of the four central nucleotides of the I-CreI target site. Despite the absence of base specific protein-DNA interactions in this region, in vitro selection of cleavable I-CreI targets from a library of randomly mutagenized sites revealed the importance of these 4 base-pairs for cleavage activity (Argast et al., J. Mol. Biol., 1998, 280:345-353.). More generally, it is unlikely that engineered meganucleases cleaving every and any 22 by sequence could be derived from the sole I-CreI scaffold, and other proteins could be used as well, including monomeric LAGLIDADG proteins.

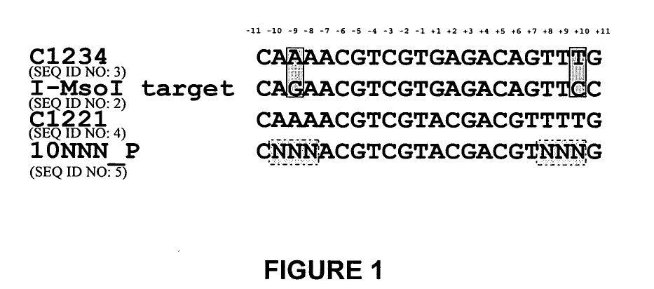

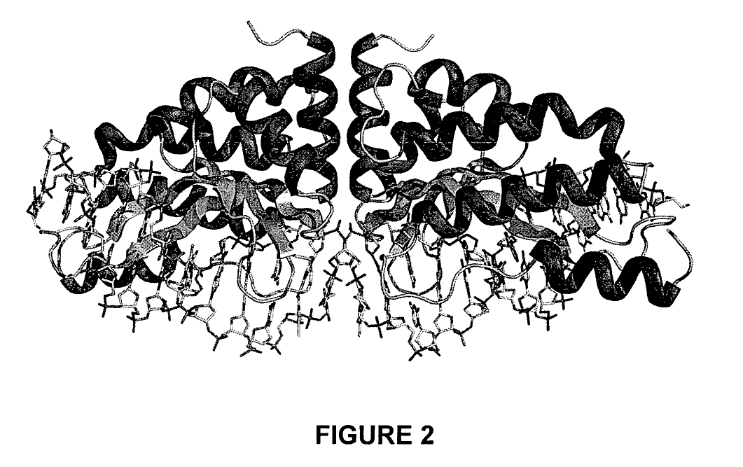

I-MsoI is an homing endonuclease from Monomastix sp. It is a homodimeric protein and it shares 36% sequence identity with I-CreI. Its DNA target is closely related to that of I-CreI, with only two differences at positions −9 and +10 (FIG. 1). In addition, I-CreI and I-MsoI both cleave each other's DNA target, and are therefore isoschizomers (Chevalier et al., J. Mol. Biol. 2003, 329:253-69). The structure of I-MsoI in complex with its DNA target has been solved (Chevalier et al., J. Mol. Biol., 2003, 329:253-269) and is shown in FIG. 2. Structure analysis showed that in spite of DNA target similarity, DNA recognition by I-MsoI and I-CreI depend on a different sets of interaction patterns.

A single I-MsoI variants (K28L, T83R) with novel cleavage specificity for positions ±6 was designed by using a pure rational process, relying on a computational approach (Ashworth et al., Nature, 2006, 441:656-659).

Computational models were used to identify specific amino acid residues that specifically interact with the I-MsoI site and predict specific amino acid substitutions which alter the specificity towards individual bases within the I-MsoI site sequence (International PCT Application WO 2007/047859). According to these predictions, the specificity towards the nucleotides at positions ±8, ±9 and ±10 of the I-MsoI site might be changed by specific substitutions of I30, S43 and I85 (position ±8), Q41 and R32 (position ±9), and Y35 and R32 (position ±10), respectively (Table 2 page 41 of WO 2007/047859). However, this approach was not validated experimentally and no I-MsoI variant having the predicted mutations was shown to have indeed a modified cleavage specificity towards the nucleotides at positions ±8, ±9 and ±10 of the I-MsoI site.

By using a semi-rational approach very similar to the one previously described to engineer the I-CreI protein, the inventors have engineered around one hundred of novel I-MsoI variants which, altogether, target 31 mutant DNA target sites differing at positions ±10, ±9, and ±8.

These variants have mutations at position 32 and/or 41 of I-MsoI sequence which are different to those predicted in the International PCT Application WO 2007/047859. Furthermore, the inventors have demonstrated that contrary to what is stated in Table 2 of WO 2007/047859, there is no correlation between a specific amino acid residue at position 32 and 41 and a particular nucleotide g, t, a or c at position ±10, ±9, and ±8. These results indicate that although, the structure of 1-MsoI in complex with its DNA target has been solved, changing the specificity of MsoI is a complex problem.

These variants having new substrate specificity towards nucleotides ±8, ±9, and/or ±10, increase the number of DNA sequences that can be targeted with meganucleases. Potential applications include genetic engineering, genome engineering, gene therapy and antiviral therapy.

Thus, the invention concerns a method for engineering a I-MsoI homing endonuclease variant having novel substrate specificity, comprising:

(a) constructing a library of I-MsoI variants having amino acid variation at one or more positions of I-MsoI amino acid sequence selected from the group consisting of: P31, R32, P33, Y35, Q41 and S43, and

(b) assaying the cleavage activity of the variants from step (a) towards a panel of DNA targets consisting of mutant I-MsoI sites wherein one or more nucleotides at positions ±8 to 10 have been replaced with different nucleotides, and

(c) selecting/screening the variants from step (b) having a pattern of cleaved DNA targets that is different from that of the parent I-MsoI homing endonuclease.

### DEFINITIONS

Amino acid residues in a polypeptide sequence are designated herein according to the one-letter code, in which, for example, P means Pro or Proline residue, R means Arg or Arginine residue and Y means Tyr or Tyrosine residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp.
by “homodimeric LAGLIDADG homing endonuclease” is intended a wild-type homodimeric LAGLIDADG homing endonuclease having a single LAGLIDADG motif and cleaving palindromic DNA target sequences, such as I-CreI or I-MsoI or a functional variant thereof.

by “I-MsoI” is intended the wild-type I-MsoI having the sequence pdb accession code 1M5X_A or 1M5X_B (SEQ ID NO: 1).
by “I-MsoI homing endonuclease variant”, “meganuclease variant” or “variant” is intended a protein obtained by replacing at least one amino acid of 1-MsoI sequence with a different amino acid. According to the invention, the amino acid residue which is mutated is indicated by its position in I-MsoI sequence SEQ ID NO: 1. For example, P31 refers to the proline residue at position 31 of the sequence SEQ ID NO: 1.
by “functional variant” is intended a I-MsoI homing endonuclease variant which is able to cleave a DNA target, preferably a new DNA target which is not cleaved by I-MsoI. For example, such variants have amino acid variation at positions interacting directly or indirectly with the DNA target sequence.
by “parent I-MsoI homing endonuclease” is intended I-MsoI or a functional variant thereof. Said parent I-MsoI homing endonuclease is a dimer (homodimer or heterodimer) comprising two I-MsoI homing endonuclease monomers/core domains which are associated in a functional endonuclease able to cleave a double-stranded DNA target of 22 to 24 bp.
by “homing endonuclease variant with novel specificity” is intended a variant having a pattern of cleaved DNA targets (cleavage profile) different from that of the parent homing endonuclease. The variants may cleave less targets (restricted profile) or more targets than the parent homing endonuclease. Preferably, the variant is able to cleave at least one target that is not cleaved by the parent homing endonuclease.

The terms “novel specificity”, “modified specificity”, “altered specificity”, “novel cleavage specificity”, “novel substrate specificity” which are equivalent and used indifferently, refer to the specificity of the variant towards the nucleotides of the DNA target sequence.
by “homing endonuclease domain”, “domain” or “core domain” is intended the “LAGLIDADG homing endonuclease core domain” which is the characteristic α₁β₁β₂α₂β₃β₄α₃ fold of the homing endonucleases of the LAGLIDADG family corresponding to a sequence of about one hundred amino acid residues. Said domain comprises four beta-strands (β₁, β₂, β₃, β₄) folded in an antiparallel beta-sheet which interacts with one half of the DNA target of a homing endonuclease and is able to associate with the other domain of the same homing endonuclease which interacts with the other half of the DNA target to form a functional endonuclease able to cleave said DNA target. For example, in the case of the dimeric homing endonuclease I-MsoI (170 amino acids), the LAGLIDADG homing endonuclease core domain corresponds to the residues 9 to 97.
by “subdomain” is intended the region of a LAGLIDADG homing endonuclease core domain which interacts with a distinct part of a homing endonuclease DNA target half-site. Two different subdomains behave independently and the mutation in one subdomain does not alter the binding and cleavage properties of the other subdomain. Therefore, two subdomains bind distinct part of a homing endonuclease DNA target half-site.
by “beta-hairpin” is intended two consecutive beta-strands of the antiparallel beta-sheet of a LAGLIDADG homing endonuclease core domain ((β₁β₂ or, β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”; “site of interest”; “recognition site”, “recognition sequence”, “homing recognition site”, “homing site”, “cleavage site” is intended a 20 to 24 by double-stranded palindromic, partially palindromic (pseudo-palindromic) or non-palindromic polynucleotide sequence that is recognized and cleaved by a LAGLIDADG homing endonuclease such as I-MsoI, or a variant, or a single-chain chimeric meganuclease derived from I-MsoI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide. Cleavage of the DNA target occurs at the nucleotides at positions +2 and −2, respectively for the sense and the antisense strand. Unless otherwise indicated, the position at which cleavage of the DNA target by an I-MsoI meganuclease variant occurs, corresponds to the cleavage site on the sense strand of the DNA target.
by “I-MsoI site” is intended a 22 to 24 by double-stranded DNA sequence which is cleaved by I-MsoI. I-MsoI sites include the wild-type (natural) non-palindromic I-MsoI homing site (SEQ ID NO: 2; FIG. 1), the I-CreI homing site (SEQ ID NO: 3) and the derived palindromic sequences which are presented in FIG. 1, such as the sequence 5′-c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₉t₊₁₀g₊₁₁ also called C1221 (SEQ ID NO: 4).
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of a different half of two parent meganuclease target sequences. In addition at least one half of said target may comprise the combination of nucleotides which are bound by at least two separate subdomains (combined DNA target).
by “vector” is intended a nucleic acid molecule capable of transporting another nucleic acid to which it has been linked.
by “homologous” is intended a sequence with enough identity to another one to lead to a homologous recombination between sequences, more particularly having at least 95% identity, preferably 97% identity and more preferably 99%.
“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.
“genetic disease” refers to any disease, partially or completely, directly or indirectly, due to an abnormality in one or several genes. Said abnormality can be a mutation, an insertion or a deletion. Said mutation can be a punctual mutation. Said abnormality can affect the coding sequence of the gene or its regulatory sequence. Said abnormality can affect the structure of the genomic sequence or the structure or stability of the encoded mRNA. Said genetic disease can be recessive or dominant. Such genetic disease could be, but are not limited to, cystic fibrosis, Huntington's chorea, familial hyperchoiesterolemia (LDL receptor defect), hepatoblastoma, Wilson's disease, congenital hepatic porphyrias, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, Duchenne's muscular dystrophy, and Tay-Sachs disease.
by mutation is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

According, to an advantageous embodiment of said method, the library in step a) comprises the replacement of the initial amino acid(s) with S, P, T, A, Y, H, Q, N, K, D, E, C, W, R and G.

The library in step (a) is prepared according to standard methods which are well-known in the art. For example, the library may be produced by amplifying fragments overlapping in the region of the mutation(s) with degenerated primer(s) to allow degeneracy at the position(s) of the mutation(s).

According to an advantageous embodiment of said method, the library in step (a) is a combinatorial library having diversity at two or three positions of I-MsoI sequence. For example, the library has diversity at positions 32 and 41, 32 and 43, 32 and 35, 32, 41 and 43, or 31, 32 and 33. Combinatorial libraries may be generated as described in International PCT Applications WO 2004/067736, WO 2006/097853, WO 2007/057781 and WO 2007/049156; Arnould et al., J. Mol. Biol., 2006, 355, 443-458; Smith et al., Nucleic Acids Res., 2006, 34, e149.

The parent I-MsoI homing endonuclease (initial scaffold protein) which is used for preparing the library of variants may be I-MsoI, for example the sequence SEQ ID NO: 1 or a functional variant of I-MsoI variant as defined above. In addition, one or more residues may be inserted at the NH₂ terminus and/or COOH terminus of the scaffold protein. Additional codons may be added at the 5′ or 3′ end of the I-MsoI coding sequence to introduce restrictions sites which are used for cloning into various vectors. An example of said sequence is SEQ ID NO: 105 which has an alanine (A) residue inserted after the first methionine residue and an alanine and an aspartic acid (AD) residues inserted after the C-terminal proline residue. These sequences allow having DNA coding sequences comprising the NcoI (ccatgg) and EagI (cggccg) restriction sites which are used for cloning into various vectors. A tag (epitope or polyhistidine sequence) may also be introduced at the NH₂ terminus and/or COOH terminus; said tag is useful for the detection and/or the purification of the meganuclease.

According to the method of the invention, the library of variants from step (a) may comprise additional mutations in order to improve the binding and/or cleavage activity of the mutants towards the DNA target(s) of interest. Said mutations may be at other positions in direct or indirect (via a water molecule) interaction with the phosphate backbone or with the nucleotide bases of the DNA target. Furthermore, random mutations may also be introduced on the whole variant or in part of the variant, in order to improve the binding and/or cleavage activity of the variant towards the DNA target(s) of interest. This may be performed by generating random mutagenesis libraries on a pool of variants, according to standard mutagenesis methods which are well-known in the art and commercially available. The additional mutations (random or site-specific) and the mutation(s) of P31, R32, P33, Y35, Q41 and/or S43 may be introduced simultaneously or subsequently.

According to the method of the invention, the DNA target in step b) may be palindromic, non-palindromic or pseudo-palindromic. Preferably, the DNA target in step b) is a palindromic target comprising the sequence: c₋₁₁n₋₁₀n₋₉n₋₈a₋₇c₋₆g₋₅t₋₄c₋₃g₋₂t₋₁a₊₁c₊₂g₊₃a₊₄c₊₅g₊₆t₊₇n₊₈n₊₉n₊₁₀g₊₁₁, wherein n is a, t, c, or g (SEQ ID NO: 5); this target derives from C1221 (SEQ ID NO: 4, FIG. 1).

According to the method of the invention, step (b) may be performed by using a cleavage assay in vitro or in vivo, as described in the International PCT Application WO 2004/067736. Preferably, step (b) is performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break. For example, the cleavage activity of the I-MsoI variant of the invention may be measured by a direct repeat recombination assay, in yeast or mammalian cells, using a reporter vector, as described in the PCT Application WO 2004/067736. The reporter vector comprises two truncated, non-functional copies of a reporter gene (direct repeats) and a DNA target sequence within the intervening sequence, cloned in a yeast or a mammalian expression vector. The DNA target sequence is palindromic and derived from a I-MsoI site such as C1221, by substitution of one to three nucleotides at positions ±8 to 10 (FIG. 1). Expression of a functional I-MsoI variant which is able to cleave the DNA target sequence, induces homologous recombination between the direct repeats, resulting in a functional reporter gene, whose expression can be monitored by appropriate assay.

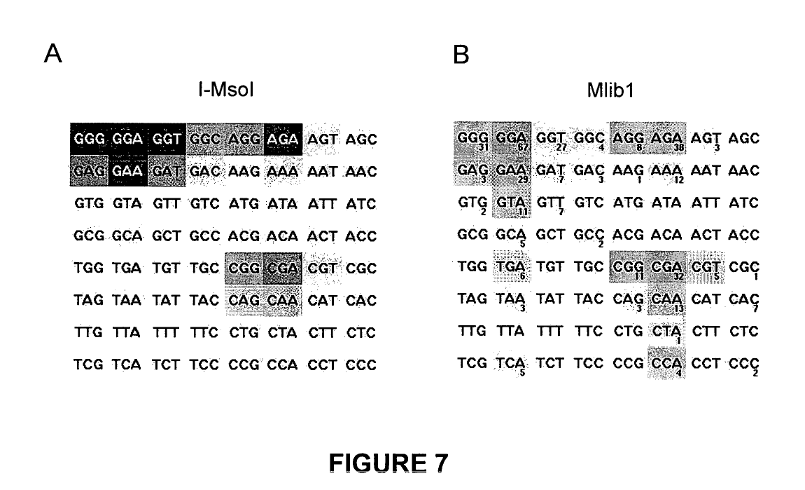

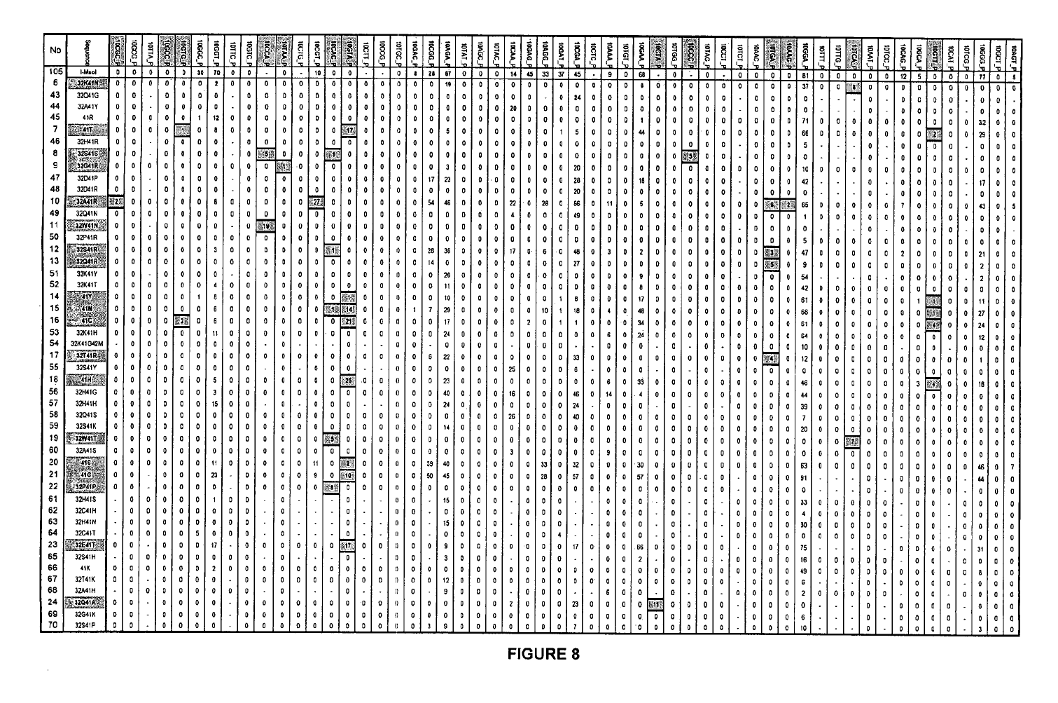

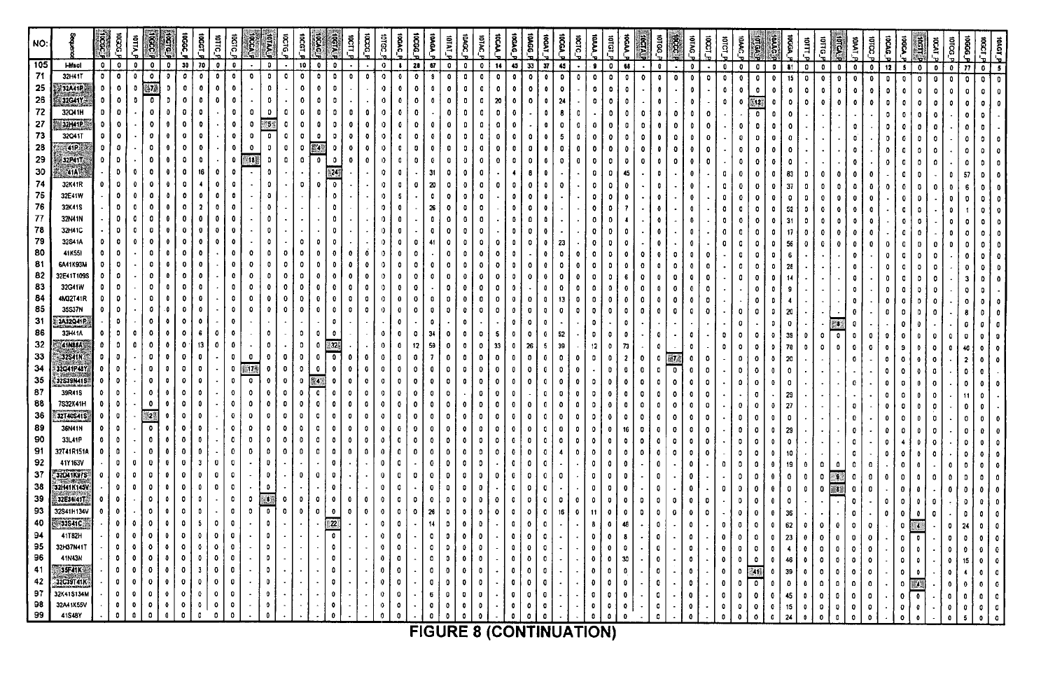

According to another advantageous embodiment of said method, step (c) comprises the selection of variants able to cleave at least one DNA target that is not cleaved by I-MsoI. The 18 targets which are cleaved by I-MsoI are presented in FIGS. 7 and 8.

According to another advantageous embodiment of said method, it comprises a further step d₁) of expressing one variant obtained in step c), so as to allow the formation of homodimers.

According to another advantageous embodiment of said method, it comprises a further step d₂) of co-expressing one variant obtained in step c) and I-MsoI or a functional variant thereof, so as to allow the formation of heterodimers. Preferably, two different variants obtained in step c) are co-expressed.

For example, host cells may be modified by one or two recombinant expression vector(s) encoding said variant(s). The cells are then cultured under conditions allowing the expression of the variant(s) and the homodimers/heterodimers which are formed are then recovered from the cell culture.

According to the method of the invention, single-chain chimeric meganucleases may be constructed by the fusion of one monomer/domain variant obtained in step (c) with a homing endonuclease domain/monomer. Said monomer/domain from a wild-type LAGLIDADG homing endonuclease or a functional variant thereof. Preferably, the two domain(s)/monomer(s) are connected by a peptidic linker. More preferably, the single-chain meganuclease comprises two monomers, each from a different variant obtained in step (c); said single-chain meganuclease is able cleave a non-palindromic chimeric target comprising one different half of each variant DNA target.

Methods for constructing single-chain chimeric meganucleases derived from homing endonucleases are well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric meganucleases derived from the variants as defined in the present invention.

The subject matter of the present invention is also a I-MsoI homing endonuclease variant obtainable by the method as defined above, said variant having at least one mutation at position 31, 32, 33, 35, 41, and/or 43 of I-MsoI, and a cleavage pattern towards a panel of mutant I-MsoI sites having variation at positions ±8 to 10, that is different from that of I-MsoI.

According to an advantageous embodiment of said I-MsoI variant, it comprises at least the replacement of Q41 with N, G, Y, R, T, S, P, C, H, K, A or W. Preferably Q41 is replaced with N, G, Y, T, S, P, C, H, A or W.

According to another advantageous embodiment of said I-MsoI variant, it comprises at least the replacement of R32 with K, Q, A, H, S, G, D, W, P, T, C, E and N. Preferably R32 is replaced with Q, A, H, S, G, D, W, P, T, C, and N.

According to another advantageous embodiment of said I-MsoI variant, it comprises at least the replacement of P31 or P33 with S, T, A, Y, H, Q, N, K, D, E, C, W, R or G.

According to another advantageous embodiment of said I-MsoI variant, it comprises at least the replacement of Y35 with S, P, T, A, H, Q, N, D, E, C, W, or G.

According to another advantageous embodiment of said I-MsoI variant, it comprises at least the replacement of S43 with P, T, A, Y, H, N, D, C, W, or G.

According to another advantageous embodiment of said I-MsoI variant, it comprises at least one additional mutation at a position of I-MsoI that improves the binding and/or the cleavage activity towards the DNA target, said position being selected from the group consisting of: T3, K4, T6, L7, K36, D37, K39, Y40, V42, F48, F55, Y82, T88, I93, L97, N109, I134, A145, T151 and A163. Preferably, said mutation is selected from the group consisting of: T3A, K4M, T6A, L7S, K36N, K36I, D37N, K39N, K39R, K39T, Y40S, V42M, F48Y, F55V, F55I, Y82H, T88A, I93M, L97S, N109S, I134V, I134M, A145V, T151A and A163V.

The invention includes a first series of I-MsoI variants able to cleave at least one DNA target having variation at positions ±8 to 10, that is not cleaved by I-MsoI, said variants comprising mutations selected from the group consisting of: R32K and Q41N; Q41T; R32S and Q41S; R32A and Q41R; R32W and Q41N; R32S and Q41R; R32Q and Q41R; Q41Y; Q41N; Q41C; R32T and Q41R; Q41H; R32W and Q41T; Q41S; Q41G; R32E and Q41T; R32Q and Q41A; R32G and Q41Y; Q41P; R32P and Q41T; Q41A; T3A, R32Q and Q41P; Q41N and T88A; R32S and Q41N; R32Q, Q41P and F48Y; R32S, K39N and Q41S; R32D, Q41K and L97S; R32H, Q41K and A145V; P33S and Q41C; Y35F and Q41K; R32C, K39T and Q41K; R32A and Q41P; R32T, Y40S and Q41S; R32G and Q41R; R32H and Q41P; R32E, K36E and Q41T, R32P and Q41P. Examples of said variants are the sequences SEQ ID NO: 6 to 42 (FIG. 8).

Preferably, said DNA target that is not cleaved by I-MsoI comprises a nucleotide triplet at positions −10 to −8, which is selected from the group consisting of: aag, gtg, gta, gtt, gcc, tga, taa, cac, cta, tca, cca, ccc and cgc and/or a nucleotide triplet at positions ±8 to +10, which is the reverse complementary sequence of said nucleotide triplet at positions −10 to −8.

The invention includes also a second series of I-MsoI variants having a cleavage pattern towards targets having variation at positions ±8 to 10 which is more restricted than that of I-MsoI, said variants comprising mutations selected from the group consisting of: R32Q and Q41G; R32A and Q41Y; R32H and Q41R; R32D and Q41P; R32D and Q41R; R32Q and Q41N; R32P and Q41R; R32K and Q41Y; R32K and Q41T; R32K and Q41H; R32K, Q41G and V42M; R32S and Q41Y; R32H and Q41G; R32H and Q41H; R32Q and Q41S; R32S and Q41K; R32A and Q41S; R32H and Q41S; R32C and Q41H; R32H and Q41N; R32C and Q41T; R32S and Q41H; R32T and Q41K; R32A and Q41H; R32G and Q41K; R32S and Q41P; R32H and Q41T; R32Q and Q41H; R32Q and Q41T; R32K and Q41R; R32E and Q41W; R32K and Q41S; R32N and Q41N; R32H and Q41C; R32S and Q41A; Q41K and F55I; T6A, Q41K and 193M; R32E, Q41T and N109S; R32G and Q41W; K4M, R32T and Q41R; Y35S and D37N; R32H and Q41A; K39R and Q41S; L7S, R32K and Q41H; K36N and Q41N; P33L and Q41P; R32T, Q41R and T151A; Q41Y and A163V; R32S, Q41H and I134V; Q41T and Y82H; R32H, D37N and Q41T; Q41N and P43N; R32K, Q41S and I134M; R32A, Q41K and F55V; Q41S and F48Y. Examples of said variants are the sequences SEQ ID NO: 43, 44, 46 to 65 and 67 to 99 (FIG. 8).

The I-MsoI variant of the invention may be an homodimer or an heterodimer.

According to another advantageous embodiment of said I-MsoI variant, it is an heterodimer comprising monomers from two different variants.

The subject-matter of the present invention is also a single-chain chimeric meganuclease (fusion protein) derived from an I-MsoI variant as defined above. The single-chain meganuclease may comprise two I-MsoI monomers, two I-MsoI core domains or a combination of both. Preferably, the two monomers/core domains or the combination of both, are connected by a peptidic linker.

The meganuclease of the invention includes both the meganuclease variant and the single-chain meganuclease derivative.

The subject-matter of the present invention is also a polynucleotide fragment encoding a variant or a single-chain chimeric meganuclease as defined above; said polynucleotide may encode one monomer of an homodimeric or heterodimeric variant, or two domains/monomers of a single-chain chimeric meganuclease.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of an heterodimeric variant.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferred vectors include lentiviral vectors, and particularly self inactivating lentiviral vectors.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is an heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA cleavage site as defined above.

Alternatively, the vector coding for an I-MsoI variant/single-chain meganuclease and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a).

Preferably, homologous sequences of at least 50 bp, preferably more than 100 by and more preferably more than 200 bp are used. Therefore, the targeting DNA construct is preferably from 200 pb to 6000 pb, more preferably from 1000 pb to 2000 pb. Indeed, shared DNA homologies are located in regions flanking upstream and downstream the site of the break and the DNA sequence to be introduced should be located between the two arms. The sequence to be introduced is preferably a sequence which repairs a mutation in the gene of interest (gene correction or recovery of a functional gene), for the purpose of genome therapy. Alternatively, it can be any other sequence used to alter the chromosomal DNA in some specific way including a sequence used to modify a specific sequence, to attenuate or activate the gene of interest, to inactivate or delete the gene of interest or part thereof, to introduce a mutation into a site of interest or to introduce an exogenous gene or part thereof. Such chromosomal DNA alterations are used for genome engineering (animal models/human recombinant cell lines).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or part of their cells are modified by a polynucleotide or a vector as defined above.

As used herein, a cell refers to a prokaryotic cell, such as a bacterial cell, or eukaryotic cell, such as an animal, plant or yeast cell.

The subject-matter of the present invention is further the use of a meganuclease, one or two derived polynucleotide(s), preferably included in expression vector(s), a cell, a transgenic plant, a non-human transgenic mammal, as defined above, for molecular biology, for in vivo or in vitro genetic engineering, and for in vivo or in vitro genome engineering, for non-therapeutic purposes.

Molecular biology includes with no limitations, DNA restriction and DNA mapping. Genetic and genome engineering for non therapeutic purposes include for example (i) gene targeting of specific loci in cell packaging lines for protein production, (ii) gene targeting of specific loci in crop plants, for strain improvements and metabolic engineering, (iii) targeted recombination for the removal of markers in genetically modified crop plants, (iv) targeted recombination for the removal of markers in genetically modified microorganism strains (for antibiotic production for example).

According to an advantageous embodiment of said use, it is for inducing a double-strand break in a site of interest comprising a DNA target sequence, thereby inducing a DNA recombination event, a DNA loss or cell death.

According to the invention, said double-strand break is for: repairing a specific sequence, modifying a specific sequence, restoring a functional gene in place of a mutated one, attenuating or activating an endogenous gene of interest, introducing a mutation into a site of interest, introducing an exogenous gene or a part thereof, inactivating or detecting an endogenous gene or a part thereof, translocating a chromosomal arm, or leaving the DNA unrepaired and degraded.

The subject-matter of the present invention is also a method of genetic engineering, characterized in that it comprises a step of double-strand nucleic acid breaking in a site of interest located on a vector comprising a DNA target as defined hereabove, by contacting said vector with a meganuclease as defined above, thereby inducing an homologous recombination with another vector presenting homology with the sequence surrounding the cleavage site of said meganuclease.

The subjet-matter of the present invention is also a method of genome engineering, characterized in that it comprises the following steps: 1) double-strand breaking a genomic locus comprising at least one DNA target of a meganuclease as defined above, by contacting said target with said meganuclease; 2) maintaining said broken genomic locus under conditions appropriate for homologous recombination with a targeting DNA construct comprising the sequence to be introduced in said locus, flanked by sequences sharing homologies with the targeted locus.

The subject-matter of the present invention is also a method of genome engineering, characterized in that it comprises the following steps: 1) double-strand breaking a genomic locus comprising at least one DNA target of a meganuclease as defined above, by contacting said cleavage site with said meganuclease; 2) maintaining said broken genomic locus under conditions appropriate for homologous recombination with chromosomal DNA sharing homologies to regions surrounding the cleavage site.

The subject-matter of the present invention is also the use of at least one meganuclease as defined above, one or two derived polynucleotide(s), preferably included in expression vector(s), as defined above, for the preparation of a medicament for preventing, improving or curing a genetic disease in an individual in need thereof, said medicament being administrated by any means to said individual.

The subject-matter of the present invention is also a method for preventing, improving or curing a genetic disease in an individual in need thereof, said method comprising the step of administering to said individual a composition comprising at least a meganuclease as defined above, by any means.

In this case, the use of the meganuclease as defined above, comprises at least the step of (a) inducing in somatic tissue(s) of the individual a double stranded cleavage at a site of interest of a gene comprising at least one recognition and cleavage site of said meganuclease, and (b) introducing into the individual a targeting DNA, wherein said targeting DNA comprises (1) DNA sharing homologies to the region surrounding the cleavage site and (2) DNA which repairs the site of interest upon recombination between the targeting DNA and the chromosomal DNA. The targeting DNA is introduced into the individual under conditions appropriate for introduction of the targeting DNA into the site of interest.

According to the present invention, said double-stranded cleavage is induced, either in toto by administration of said meganuclease to an individual, or ex vivo by introduction of said meganuclease into somatic cells removed from an individual and returned into the individual after modification.

In a preferred embodiment of said use, the meganuclease is combined with a targeting DNA construct comprising a sequence which repairs a mutation in the gene flanked by sequences sharing homologies with the regions of the gene surrounding the genomic DNA cleavage site of said meganuclease, as defined above. The sequence which repairs the mutation is either a fragment of the gene with the correct sequence or an exon knock-in construct.

For correcting a gene, cleavage of the gene occurs in the vicinity of the mutation, preferably, within 500 bp of the mutation. The targeting construct comprises a gene fragment which has at least 200 by of homologous sequence flanking the genomic DNA cleavage site (minimal repair matrix) for repairing the cleavage, and includes the correct sequence of the gene for repairing the mutation. Consequently, the targeting construct for gene correction comprises or consists of the minimal repair matrix; it is preferably from 200 pb to 6000 pb, more preferably from 1000 pb to 2000 pb.

For restoring a functional gene, cleavage of the gene occurs upstream of a mutation. Preferably said mutation is the first known mutation in the sequence of the gene, so that all the downstream mutations of the gene can be corrected simultaneously. The targeting construct comprises the exons downstream of the genomic DNA cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein. For example, the exon knock-in construct is flanked by sequences upstream and downstream.

The subject-matter of the present invention is also the use of at least one meganuclease as defined above, one or two derived polynucleotide(s), preferably included in expression vector(s), as defined above for the preparation of a medicament for preventing, improving or curing a disease caused by an infectious agent that presents a DNA intermediate, in an individual in need thereof, said medicament being administrated by any means to said individual.

The subject-matter of the present invention is also a method for preventing, improving or curing a disease caused by an infectious agent that presents a DNA intermediate, in an individual in need thereof, said method comprising at least the step of administering to said individual a composition as defined above, by any means.

The subject-matter of the present invention is also the use of at least one meganuclease as defined above, one or two polynucleotide(s), preferably included in expression vector(s), as defined above, in vitro, for inhibiting the propagation, inactivating or deleting an infectious agent that presents a DNA intermediate, in biological derived products or products intended for biological uses or for disinfecting an object.

The subject-matter of the present invention is also a method for decontaminating a product or a material from an infectious agent that presents a DNA intermediate, said method comprising at least the step of contacting a biological derived product, a product intended for biological use or an object, with a composition as defined above, for a time sufficient to inhibit the propagation, inactivate or delete said infectious agent.

In a particular embodiment, said infectious agent is a virus. For example said virus is an adenovirus (Ad11, Ad21), herpesvirus (HSV, VZV, EBV, CMV, herpesvirus 6, 7 or 8), hepadnavirus (HBV), papovavirus (HPV), poxvirus or retrovirus (HTLV, HIV).

The subject-matter of the present invention is also a composition characterized in that it comprises at least one meganuclease, one or two derived polynucleotide(s), preferably included in expression vector(s), as defined above.

In a preferred embodiment of said composition, it comprises a targeting DNA construct comprising the sequence which repairs the site of interest flanked by sequences sharing homologies with the targeted locus as defined above. Preferably, said targeting DNA construct is either included in a recombinant vector or it is included in an expression vector comprising the polynucleotide(s) encoding the meganuclease, as defined in the present invention.

The subject-matter of the present invention is also products containing at least a meganuclease, or one or two expression vector(s) encoding said meganuclease, and a vector including a targeting construct, as defined above, as a combined preparation for simultaneous, separate or sequential use in the prevention or the treatment of a genetic disease.

For purposes of therapy, the meganuclease and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the meganuclease is substantially non-immunogenic, i.e., engenders little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

The meganuclease can be used either as a polypeptide or as a polynucleotide construct/vector encoding said polypeptide. It is introduced into cells, in vitro, ex vivo or in vivo, by any convenient means well-known to those in the art, which are appropriate for the particular cell type, alone or in association with either at least an appropriate vehicle or carrier and/or with the targeting DNA. Once in a cell, the meganuclease and if present, the vector comprising targeting DNA and/or nucleic acid encoding a meganuclease are imported or translocated by the cell from the cytoplasm to the site of action in the nucleus.

The meganuclease (polypeptide) may be advantageously associated with: liposomes, polyethyleneimine (PEI), and/or membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in the latter case, the sequence of the meganuclease fused with the sequence of a membrane translocating peptide (fusion protein).

Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

The subject-matter of the present invention is also the use of at least one meganuclease, as defined above, as a scaffold for making other meganucleases. For example other rounds of mutagenesis and selection/screening can be performed on the variant, for the purpose of making novel homing endonucleases.

The uses of the meganuclease and the methods of using said meganucleases according to the present invention include also the use of the polynucleotide(s), vector(s), cell, transgenic plant or non-human transgenic mammal encoding said meganuclease, as defined above.

According to another advantageous embodiment of the uses and methods according to the present invention, said meganuclease, polynucleotide(s), vector(s), cell, transgenic plant or non-human transgenic mammal are associated with a targeting DNA construct as defined above. Preferably, said vector encoding the monomer(s) of the meganuclease, comprises the targeting DNA construct, as defined above.

The polynucleotide fragments having the sequence of the targeting DNA construct or the sequence encoding the meganuclease variant or single-chain meganuclease derivative as defined in the present invention, may be prepared by any method known by the man skilled in the art. For example, they are amplified from a DNA template, by polymerase chain reaction with specific primers. Preferably the codons of the cDNAs encoding the meganuclease variant or single-chain meganuclease derivative are chosen to favour the expression of said proteins in the desired expression system.

The recombinant vector comprising said polynucleotides may be obtained and introduced in a host cell by the well-known recombinant DNA and genetic engineering techniques.

The meganuclease variant or single-chain meganuclease derivative as defined in the present the invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the meganuclease variant or single-chain meganuclease derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In addition to the preceding features, the invention further comprises other features which will emerge from the description which follows, which refers to examples illustrating the I-MsoI homing endonuclease variants and their uses according to the invention, as well as to the appended drawings in which:

FIG. 1 represents the DNA targets. The C1234 wild-type I-CreI target and I-MsoI target are close derivatives: the two differences between the two targets have been boxed in grey. They were first described as 24 by sequences but structural data indicate that only 22 by are relevant for protein/DNA interaction. C1221 is the palindromic sequence derived from the left part of C1234. A 10NNN_P target is a derivative from C1221, where a degeneracy at positions ±10, ±9, ±8 has been introduced.

FIG. 2 represents the structure of the I-MsoI homing endonuclease in complex with its DNA target according to Chevalier et al., J. Mol. Biol., 2003, 329, 253-269 (PDB code 1M5X).

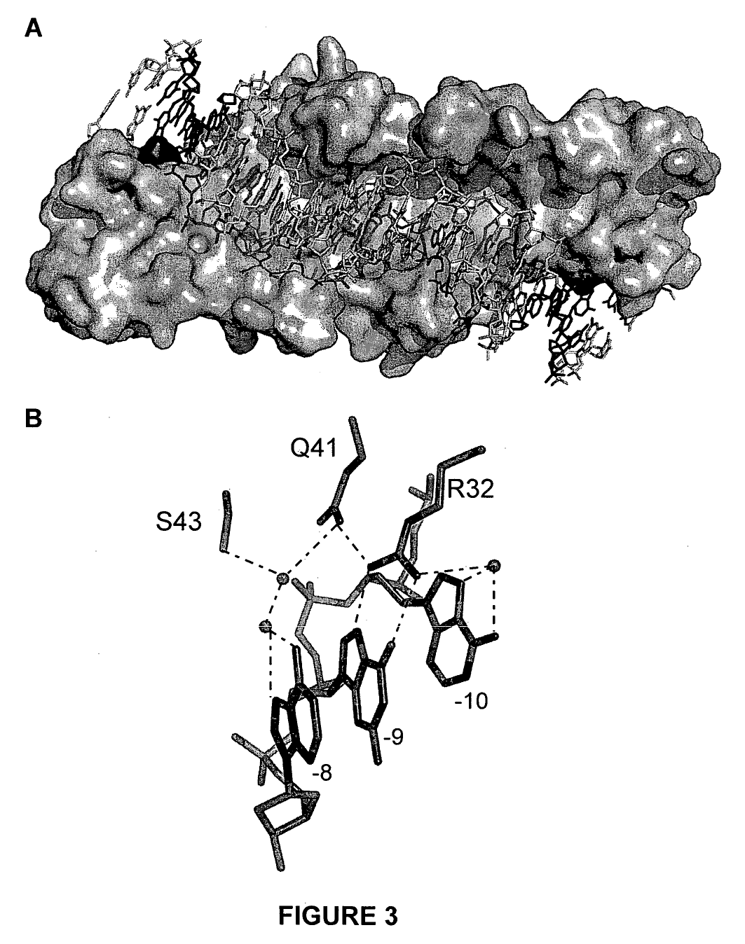

FIG. 3 represents the area of the binding interface chosen for randomization in this study. A. Molecular surface of I-MsoI bound to its DNA target: base pairs at positions ±10, ±9, ±8 and protein residues 32, 41 and 43 chosen for randomization are labeled in black. B. Zoom showing residues 32, 41 and 43 in interaction with the nucleotides −10, −9 and −8 of the DNA target. Grey spheres are water molecules and dashed lines represent hydrogen bonds.

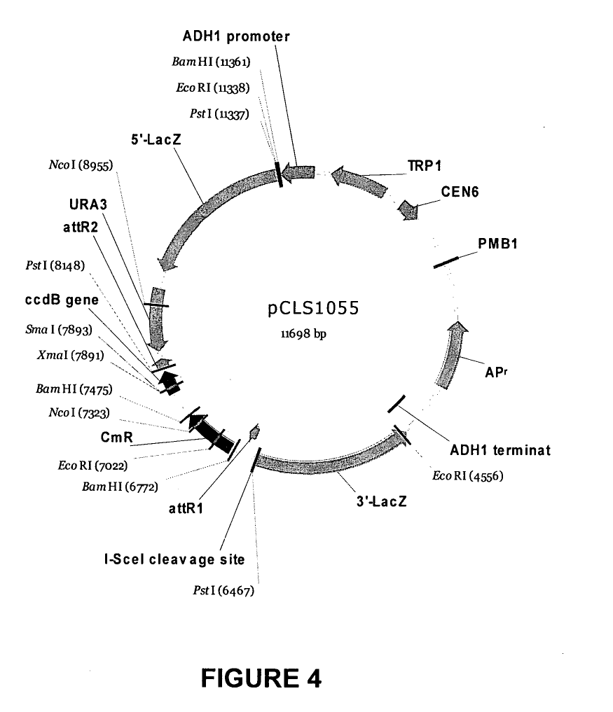

FIG. 4 represents the pCLS1055 reporter vector map. The reporter vector is marked with TRP1 and URA3. The LacZ tandem repeats share 800 by of homology, and are separated by 1.3 kb of DNA. They are surrounded by ADH promoter and terminator sequences. Target sites are cloned using the Gateway protocol (Invitrogen), resulting in the replacement of the CmR and ccdB genes with the chosen target site.

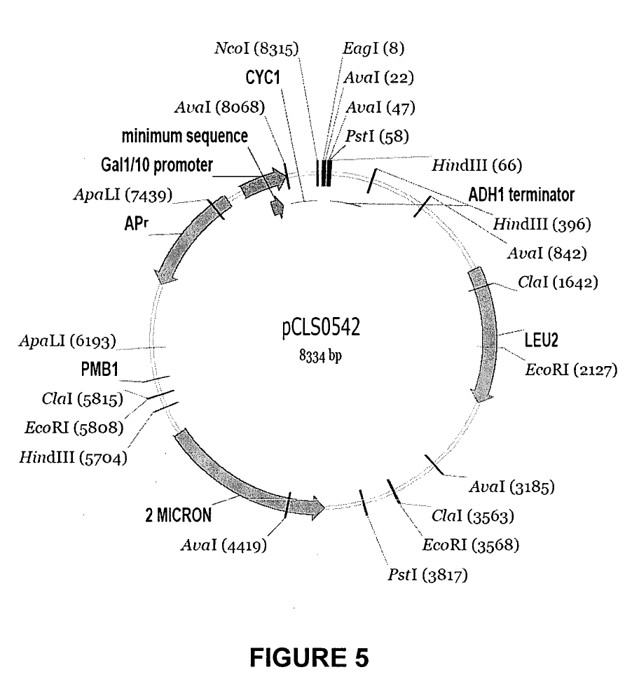

FIG. 5 represents the pCLS0542 meganuclease expression vector map. pCLS0542 is a 2 micron-based replicative vector marked with a LEU2 auxotrophic gene, and an inducible Gal10 promoter for driving the expression of the I-MsoI variants.

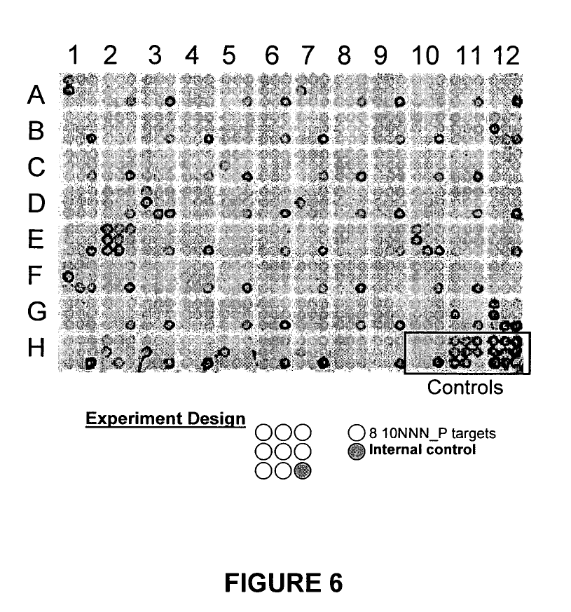

FIG. 6 displays an example of primary screening of I-MsoI mutants from the Mlib1 library against 8 10NNN_P targets. Columns and rows are respectively noted from 1 to 12 and from A to H. In each 9-dots yeast cluster, a Mlib1 mutant is screened against 8 different targets as exemplified by the experimental design. The bottom right dot is a cluster internal control. Depending on the cluster, it is either a negative control (no meganuclease) either a positive control (weak or strong versions of I-SceI, assayed on I-SceI target). H10, H11 and H12 are also experiment controls.

FIG. 7 displays the hitmap of I-MsoI and I-MsoI variants against the 64 10NNN_P targets. A. I-MsoI hitmap. B. Mlib1 library hitmap. Each novel endonuclease is profiled in yeast on a series of 64 palindromic targets described in FIG. 1, differing from the sequence shown in FIG. 1, at positions ±8, ±9 and ±10. Each target sequence is named after the −10, −9, −8 triplet (10NNN). For example GGG corresponds to the cgggacgtcgtacgacgtcccg target (SEQ ID NO: 104). The number below each cleaved target is the number of I-MsoI mutants with different sequences cleaving this target. For each target, the grey level is proportional to the mean of cleavage intensity.

FIG. 8 displays represents the cleavage patterns of I-MsoI variants cleaving 31 DNA targets. For I-MsoI and each of the I-MsoI variants (SEQ ID NO: 6 to 99) obtained after screening and defined by the indicated residues, cleavage was monitored in yeast with the 64 targets described in FIG. 7. Targets are designated by three letters, corresponding to the nucleotides at position −10, −9 and −8. For example GGG corresponds to the cgggacgtcgtacgacgtcccg target (SEQ ID NO: 104; see FIG. 1). Values correspond to the intensity of the cleavage, evaluated by an appropriate software after scanning of the filter. The 13 targets which are not cleaved by I-MsoI are highlighted in grey with the corresponding variants and their cleavage score.

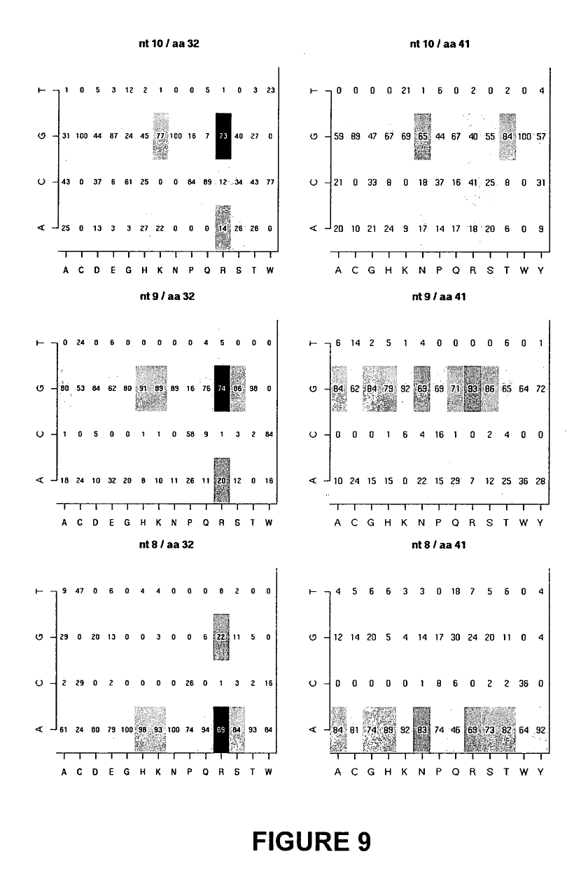

FIG. 9 illustrates the correlation between given residues at positions 32 and 41 of I-MsoI and bases at positions ±10; ±9 and ±8 (10NNN) of the target. The sum of all the intensities of cleavage from the matrix of FIG. 8 are featured as a level of grey intensity, with a cumulated intensity of 30 corresponding arbitrarily to black and 0 corresponding to white, for a mutant which has A, C, G, H, K, N, P, Q, R, S, T, W or Y at position 32 (left panel) or 41 (right panel) and tested with targets which have a, c, g or t at position −10, −9 or −8 (upper, medium and lower panel, respectively). The values are normalized to 100 by column.

### EXAMPLE 1

### Making of I-MsoI Derived Mutants Cleaving Degenerated 10NNN_P Targets

This example shows that I-MsoI mutants can cut DNA target sequences derived from the C1221 target, a target efficiently cleaved by I-CreI and I-MsoI, and shown in FIG. 1. I-MsoI residues in direct or indirect interaction with the DNA target nucleotides at position ±10; ±9 and ±8 (10NNN) were pintpointed by a close examination of the structure displayed in FIG. 2. By direct interaction is meant a hydrogen bond between a protein residue and a base pair, an indirect interaction being a water-mediated interaction between the protein and the DNA. For example, the residue R32 makes two hydrogen bonds with the guanine at position −9 and contacts a water molecule, which itself interacts with the adenine at position −10. Q41 and S43 are connected to the adenine at position −8 via a water molecules network (FIG. 3). In order to isolate new cleavage specificities for the I-MsoI protein, an I-MsoI mutant library mutated at positions 32 and 41 (Mlib1) was built, transformed in the yeast and screened against the 64 degenerated palindromic 10NNN_P targets (see FIG. 1) using the previously described screening assay based on cleavage-induced recombination in yeast cells (International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458). These assay results in a functional LacZ reporter gene which can be monitored by standard methods. Such an approach has been already thoroughly described for the I-CreI protein (Smith et al., Nucleic Acids Res., 2006, 34, e149; International PCT Application WO 2007/049156).

### 1) Material and Methods

### a) Construction of the 64 Target Vectors

The targets were cloned as follows: oligonucleotides corresponding to each of the 64 target sequences flanked by gateway cloning sequence were ordered from PROLIGO: 5′ tggcatacaagtttcnnnacgtcgtacgacgtnnngacaatcgtctgtca 3′ (SEQ ID NO: 100). Double-stranded target DNA, generated by PCR amplification of the single stranded oligonucleotide, was cloned using the Gateway protocol (INVITROGEN) into yeast reporter vector (pCLS1055, FIG. 4). Yeast reporter vector was transformed into S. cerevisiae strain FYBL2-7B (MAT a, ura3Δ851, trp1Δ63, leu2Δ1, lys2Δ202) using a high efficiency LiAc transformation protocol (Gietz and Woods, Methods Enzymol., 2002, 350, 87-96).

### b) Construction of the I-MsoI Mlib1 Mutant Library:

In order to generate I-MsoI derived coding sequences containing mutations at positions 32 and 41, separate overlapping PCR reactions were carried out that amplify the 5′ end (aa positions 1-48) or the 3′ end (positions 44-174) of the I-MsoI coding sequence (SEQ ID NO: 105). For the 3′ end, PCR amplification is carried out using a primer specific to the vector (pCLS0542, FIG. 5) (Gal10R 5′-acaaccttgattggagacttgacc-3′: SEQ ID NO: 101) and a primer specific to the I-MsoI coding sequence for amino acids 44-56 (MlibF1 5′-ctagcaatttatttatacaaagaaaagataaatttcc-3′: SEQ ID NO: 102). For the 5′ end, PCR amplification is carried out using a primer specific to the vector pCLS0542 (Gal10F 5′-gcaactttagtgctgacacatacagg-3′: SEQ ID NO: 103) and a primer specific to the I-MsoI coding sequence for amino acids 29-48 (Mlib1R 5′-aaaagaaattgctagactcacmbnatatttaatgtctttgtaatcaggmbnaggaataag-3′(SEQ ID NO: 106). The mbn code in the oligonucleotide resulting in a NVK codon at position 32 and 41 allows the degeneracy at these positions among a group of 15 possible amino acids (S, P, T, A, Y, H, Q, N, K, D, E, C, W, R and G). Then, 25 ng of each of the two overlapping PCR fragments and 75 ng of vector DNA (pCLS0542) linearized by digestion with NcoI and EagI were used to transform the yeast Saccharomyces cerevisiae strain FYC₂₋₆A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast. The Mlib1 nucleic diversity is 24²=576, so after transformation, 1116 clones, around two times the library diversity, were picked.

### c) Mating of Meganuclease Expressing Clones and Screening in Yeast

Mating was performed using a colony gridder (QpixII, GENETIX). Mutants were gridded on nylon filters covering YPD plates, using a low gridding density (about 4 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (1%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Mutants

To recover the mutant expressing plasmids, yeast DNA was extracted using standard protocols and used to transform E. coli. Sequencing of mutant ORF was then performed on the plasmids by MILLEGEN SA. Alternatively, ORFs were amplified from yeast DNA by PCR (Akada et al., Biotechniques, 2000, 28, 668-670), and sequence was performed directly on PCR product by MILLEGEN SA.

### 2) Results

Using the yeast screening assay that has been described above, the 1116 clones that constitute the I-MsoI Mlib1 library were screened against the 64 10NNN_P targets. The screen gave 246 positive clones able to cleave at least one 10NNN_P target (FIG. 6), resulting after sequencing in 94 unique meganucleases. The I-MsoI protein is able to cleave 18 out of the 64 10NNN_P targets (FIG. 7A). The Mlib1 hitmap displayed in FIG. 7B shows that by introducing mutations at positions 32 and 41 in the I-MsoI coding sequence, 13 new additional 10NNN_P targets are now being cleaved by I-MsoI derived mutants. The cleavage pattern of the variants is described in FIG. 8. This screening approach has therefore allowed to widen the I-MsoI cleavage spectrum of 10NNN_P targets and to isolate new cleavage specificities.

### EXAMPLE 2

### Analysis of Correlation Between Given Residues at Positions 32 and 41 of I-MsoI and Bases at Positions ±10; ±9 and ±8 (10NNN) of the Target

To identify potential correlation between specific residues at positions 32 and 41 of I-MsoI and bases at positions ±10; ±9 and ±8 (10NNN) of the target, a statistical analysis of the positives was conducted.

### 1) Materials and Methods

From the initial (mutant,target) matrix, and for each pair (p, q) of mutated amino-acid position ‘p’ on the protein and nucleic acid position ‘q’ on the target, a matrix of cumulated intensities was computed from the data from FIG. 8. This matrix of cumulated intensities has a number of columns equal to the number of distinct amino-acids occurring at p on our set of mutants and 4 rows (one for each nucleotide). The value of this matrix for amino-acid value ‘A’ and nucleotide ‘N’ is the sum of all the intensities of the initial matrix for mutants which have an A at position p and tested with targets which have an N at position q. On FIG. 9, these values are featured as a level of grey intensity, with a cumulated intensity of 30 corresponding arbitrarily to black and 0 corresponding to white. Then, this matrix was normalized to 100 by column (sum of all the cells for each column equal to 100). An image corresponding to each matrix was drawn, with the normalized value written in each cell.

### 2) Results

Results are summarized in the six panels from FIG. 9. Only dark cells are significant since the brighter cells represent only low levels of cuttings.

Potentially significant correlations between amino-acids and nucleotides should correspond to amino-acids with highly differing percentage reparation along the four nucleotides while cells are dark enough to ensure significance. R32 (found in the wild type protein as well as in several mutants) and bases G, G and A at positions ±10; ±9 and ±8 of the targets are often associated, revealing the overepresentation of a nearly wild type profile (note that the sequence of the wild type target I-MsoI is AGA in −10, −9 and −8, top strand, and GAA in 8, 9, 10, bottom strand). No other significant association could be inferred from the sample of positives, showing an absence of correlation between individual residues at position 32 and 41 and bases ±10; ±9 and ±8 of the targets. Thus, one can hypothesize that specificity for each specific base is largely influenced by more than one protein residue.

1. An I-MsoI variant which has at least one substitution at least one of positions 31, 32, 33, 35, 41, and 43 of I-MsoI, wherein the at least one substitution is selected from the group consisting of:
replacement of P31 or P33 with S, T, A, Y, H, Q, N, K, D, E, C, W, R or G,
replacement of R32 with Q, A, H, S, G, D, W, P, T, C, or N.
replacement of Y35 with S, P, T, A, H, Q, N, D, E, C, W, or G,
replacement of Q41 with N, G, Y, T, S, P, C, H, A or W,
replacement of Y35 with S, T, A, H, Q, N, K, D, E, C, W, R or G, and
replacement of S43 with P, T, A, Y, H, N, D, C, W, or G, wherein said variant is able to cleave a panel of mutant I-MsoI sites having variation at positions ±8 to 10 that is different from sites cleaved by I-MsoI.

2. The variant according to claim 1, comprising at least one additional substitution at a position of I-MsoI that improves at least one of binding and cleavage activities towards a DNA target, wherein the at least one additional substitution is selected from the group consisting of. T3, K4, T6, L7, K36, D37, K39, Y40, V42, F48, F55, Y82, T88, 193, L97, N109, I134, A145, T151 and A163.

3. The variant according to claim 2, wherein said substitution is selected from the group consisting of. T3A, K4M, T6A, L7S, K36N, K361, D3N7, K39N, K39R, K39T, Y40S, V42M, F48Y, F55V, F55I, Y82H, T88A, I93M, L97S, N109S, I134V, I134M, A145V, T151A and A163V.

4. The variant according to claim 1, which is able to cleave at least one target that is not cleaved by I-MsoI, said variant comprising substitutions selected from the group consisting of. R32K and Q41N; Q41T; R32S and Q41S; R32A and Q41R; R32W and Q41N; R32S and Q41R; R32Q and Q41R; Q41Y; Q41N; Q41C; R32T and Q41R; Q41H; R32W and Q41T; Q41S; Q41G; R32E and Q41T; R32Q and Q41A; R32G and Q41Y; Q41P; R32P and Q41T; Q41A; T3A, R32Q and Q41P; Q41N and T88A; R32S and Q41N; R32Q, Q41P and F48Y; R32S, K39N and Q41S; R32D, Q41K and L97S; R32H, Q41K and A145V; P33S and Q41C; Y35F and Q41K; R32C, K39T and Q41K; R32A and Q41P; R32T, Y40S and Q41S; R32G and Q41R; R32H and Q41P; R32E, K36E and Q41T; R32P and Q41P.

5. The variant according to claim 1, which cleaves less targets than I-MsoI, said variant comprising substitutions selected from the group consisting of. R32Q and Q41G; R32A and Q41Y; R32H and Q41R; R32D and Q41P; R32D and Q41R; R32Q and Q41N; R32P and Q41R; R32K and Q41Y; R32K and Q41T; R32K and Q41H; R32K, Q41G and V42M; R32S and Q41Y; R32H and Q41G; R32H and Q41H; R32Q and Q41S; R32S and Q41K; R32A and Q41S; R32H and Q41S; R32C and Q41H; R32H and Q41N; R32C and Q41T; R32S and Q41H; R32T and Q41K; R32A and Q41H; R32G and Q41K; R32S and Q41P; R32H and Q41T; R32Q and Q41H; R32Q and Q41T; R32K and Q41R; R32E and Q41W; R32K and Q41S; R32N and Q41N; R32H and Q41C; R32S and Q41A; Q41K and F55I; T6A, Q41K and 193M; R32E, Q41T and N109S; R32G and Q41W; K4M, R32T and Q41R; Y35S and D37N; R32H and Q41A; K39R and Q41S; L7S, R32K and Q41H; K36N and Q41N; P33L and Q41P; R32T, Q41R and T151A; Q41Y and A163V; R32S, Q41H and I134V; Q41T and Y82H; R32H, D37N and Q41T; Q41N and P43N; R32K, Q41S and I134M; R32A, Q41K and F55V; Q41S and F48Y.

6. The variant according to claim 1, wherein the variant is an homodimer.

7. The variant according to claim 1, wherein the variant is an heterodimer comprising two different variants according to claim 1.

8. A single-chain chimeric meganuclease obtained from the variant according to claim 1, comprising two monomers, two core domains or a combination of one monomer and one core domain from said variant.

9. A polynucleotide fragment encoding at least one monomer of the variant of claim 1.

10. An expression vector comprising at least one polynucleotide fragment of claim 9, wherein the at least one polynucleotide fragment is operatively linked to regulatory sequences allowing production of said variant.

11. The vector of claim 10, comprising targeting DNA construct comprising sequences sharing homologies with a region surrounding a genomic DNA target sequence that is cleaved by said meganuclease variant or single-chain meganuclease.

12. The vector of claim 11, wherein said targeting DNA construct comprises. a) sequences sharing homologies with the region surrounding the genomic DNA target sequence that is cleaved by said meganuclease variant or single-chain meganuclease, and b) sequences to be introduced flanked by a).

13. A host cell comprising at least one polynucleotide fragment according to claim 9.

14. A non-human transgenic animal comprising one polynucleotide fragment according to claim 9.

15. A transgenic plant comprising at least one polynucleotide fragment according to claim 9.

16. A pharmaceutical composition comprising at least a variant of claim 1.

17. The composition of claim 16, which comprises a targeting DNA construct comprising a sequence which repairs a genomic site of interest flanked by sequences sharing homologies with a targeted locus.

18. (canceled)

19. (canceled)

20. A method of treating a disease caused by an infectious agent that presents a DNA intermediate, in an individual in need thereof, comprising administering the variant of claim 1 to the individual.

21. A method for inhibiting an infectious agent that presents a DNA intermediate, in a biological derived product intended for biological uses or for disinfecting an object, the method comprising incorporating the variant of claim 1 into the product.

22. The method according to claim 20, wherein said infectious agent is a virus.

23. The method according to claim 18, wherein said variant is associated with a targeting DNA construct.

24. A scaffold for engineering a meganuclease, comprising the variant according to claim 1.

25. A method for genetic engineering, comprising generating a double-strand break in a site of interest comprising at least one recognition and cleavage site of a variant according to claim 1, by contacting said recognition and cleavage site with said variant, wherein said double-strand break thereby induces a DNA recombination event, a DNA loss or cell death.

26. A method for preventing, improving or curing a genetic disease in an individual in need thereof, comprising administering the variant of claim 1 to the individual.


SEQUENCE LISTING (106 sequences)

SEQ ID NO: 1 - protein, 170 aa, Monomastix sp.
MTTKNTLQPTEAAYIAGFLDGDGSIYAKLIPRPDYKDIKYQVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSP

SEQ ID NO: 2 - DNA, 22 bp, Monomastix sp.
cagaacgtcgtgagacagttcc

SEQ ID NO: 3 - DNA, 22 bp, Chlamydomonas reinhardtii
caaaacgtcgtgagacagtttg

SEQ ID NO: 4 - DNA, 22 bp, artificial sequence
  I-CreI C1221 target
caaaacgtcgtacgacgttttg

SEQ ID NO: 5 - DNA, 22 bp, artificial sequence
  10 NNN_P target
cnnnacgtcgtacgacgtnnng

SEQ ID NO: 6 - protein, 173 aa, artificial sequence
  I-MsoI variant
MATTKNTLQPTEAAYIAGFLDGDGSIYAKLIPKPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 7 - protein, 173 aa, artificial sequence
  I-MsoI variant
MATTKNTLQPTEAAYIAGFLDGDGSIYAKLIPRPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 8 - protein, 173 aa, artificial sequence
  I-MsoI variant
MATTKNTLQPTEAAYIAGFLDGDGSIYAKLIPSPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 9 - protein, 173 aa, artificial sequence
  I-MsoI variant
MATTKNTLQPTEAAYIAGFLDGDGSIYAKLIPGPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 10 - protein, 173 aa, artificial sequence
  I-MsoI variant
MATTKNTLQPTEAAYIAGFLDGDGSIYAKLIPAPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 11 - protein, 173 aa, artificial sequence
  I-MsoI variant
MATTKNTLQPTEAAYIAGFLDGDGSIYAKLIPWPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 12 - protein, 173 aa, artificial sequence
  I-MsoI variant
MATTKNTLQPTEAAYIAGFLDGDGSIYAKLIPSPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 13 - protein, 173 aa, artificial sequence
  I-MsoI variant
MATTKNTLQPTEAAYIAGFLDGDGSIYAKLIPQPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 14 - protein, 173 aa, artificial sequence
  I-MsoI variant
MATTKNTLQPTEAAYIAGFLDGDGSIYAKLIPRPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 15 - protein, 173 aa, artificial sequence
  I-MsoI variant
MATTKNTLQPTEAAYIAGFLDGDGSIYAKLIPRPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 16 - protein, 173 aa, artificial sequence
  I-MsoI variant
MATTKNTLQPTEAAYIAGFLDGDGSIYAKLIPRPDYKDIKYCVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 17 - protein, 173 aa, artificial sequence
  I-MsoI variant
MATTKNTLQPTEAAYIAGFLDGDGSIYAKLIPTPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 18 - protein, 173 aa, artificial sequence
  I-MsoI variant
MATTKNTLQPTEAAYIAGFLDGDGSIYAKLIPRPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 19 - protein, 173 aa, artificial sequence
  I-MsoI variant
MATTKNTLQPTEAAYIAGFLDGDGSIYAKLIPWPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 20 - protein, 173 aa, artificial sequence
  I-MsoI variant
MATTKNTLQPTEAAYIAGFLDGDGSIYAKLIPRPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 21 - protein, 173 aa, artificial sequence
  I-MsoI variant
MATTKNTLQPTEAAYIAGFLDGDGSIYAKLIPRPDYKDIKYGVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 22 - protein, 173 aa, artificial sequence
  I-MsoI variant
MATTKNTLQPTEAAYIAGFLDGDGSIYAKLIPPPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 23 - protein, 173 aa, artificial sequence
  I-MsoI variant
MATTKNTLQPTEAAYIAGFLDGDGSIYAKLIPEPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 24 - protein, 173 aa, artificial sequence
  I-MsoI variant
MATTKNTLQPTEAAYIAGFLDGDGSIYAKLIPQPDYKDIKYAVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 25 - protein, 173 aa, artificial sequence
  I-MsoI variant
MATTKNTLQPTEAAYIAGFLDGDGSIYAKLIPAPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 26 - protein, 173 aa, artificial sequence
  I-MsoI variant
MATTKNTLQPTEAAYIAGFLDGDGSIYAKLIPGPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 27 - protein, 173 aa, artificial sequence
  I-MsoI variant
MATTKNTLQPTEAAYIAGFLDGDGSIYAKLIPHPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 28 - protein, 173 aa, artificial sequence
  I-MsoI variant
MATTKNTLQPTEAAYIAGFLDGDGSIYAKLIPRPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 29 - protein, 173 aa, artificial sequence
  I-MsoI variant
MATTKNTLQPTEAAYIAGFLDGDGSIYAKLIPPPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 30 - protein, 173 aa, artificial sequence
  I-MsoI variant
MATTKNTLQPTEAAYIAGFLDGDGSIYAKLIPRPDYKDIKYAVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 31 - protein, 173 aa, artificial sequence
  I-MsoI variant
MATAKNTLQPTEAAYIAGFLDGDGSIYAKLIPQPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 32 - protein, 173 aa, artificial sequence
  I-MsoI variant
MATTKNTLQPTEAAYIAGFLDGDGSIYAKLIPRPDYKDIKYNVSLAISFIQRKDKFPYLQDIYDQLGKRGNLRKDRGDGIADYTIIGSAHLSIILPDLVPYLRIKKKQANRILHIINLYPQAQKNPSKFLDLVKIVDDVQNLNKRADELKSTNYDRLLEEFLKAGKIESSPAD

SEQ ID NO: 33 - protein, 173 aa, artificial sequence
  I-MsoI variant
MATTKNTLQPTEAAYIAGFLDGDGSIYAKLIPSPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 34 - protein, 173 aa, artificial sequence
  I-MsoI variant
MATTKNTLQPTEAAYIAGFLDGDGSIYAKLIPQPDYKDIKYPVSLAISYIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 35 - protein, 173 aa, artificial sequence
  I-MsoI variant
MATTKNTLQPTEAAYIAGFLDGDGSIYAKLIPSPDYKDINYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 36 - protein, 173 aa, artificial sequence
  I-MsoI variant
MATTKNTLQPTEAAYIAGFLDGDGSIYAKLIPTPDYKDIKSSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 37 - protein, 173 aa, artificial sequence
  I-MsoI variant
MATTKNTLQPTEAAYIAGFLDGDGSIYAKLIPDPDYKDIKYKVSLAISFIQRKDKFPYLQDIYDQLGKRGNLRKDRGDGIADYTIIGSTHLSIILPDSVPYLRIKKKQANRILHIINLYPQAQKNPSKFLDLVKIVDDVQNLNKRADELKSTNYDRLLEEFLKAGKIESSPAD

SEQ ID NO: 38 - protein, 173 aa, artificial sequence
  I-MsoI variant
MATTKNTLQPTEAAYIAGFLDGDGSIYAKLIPHPDYKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRVDELKSTNYDRLLEEFLKAGKIESSPAD

SEQ ID NO: 39 - protein, 173 aa, artificial sequence
  I-MsoI variant
MATTKNTLQPTEAAYIAGFLDGDGSIYAKLIPEPDYIDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 40 - protein, 173 aa, artificial sequence
  I-MsoI variant
MATTKNTLQPTEAAYIAGFLDGDGSIYAKLIPRSDYKDIKYCVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 41 - protein, 173 aa, artificial sequence
  I-MsoI variant
MATTKNTLQPTEAAYIAGFLDGDGSIYAKLIPRPDFKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 42 - protein, 173 aa, artificial sequence
  I-MsoI variant
MATTKNTLQPTEAAYIAGFLDGDGSIYAKLIPCPDYKDITYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 43 - protein, 173 aa, artificial sequence
  I-MsoI variant
MATTKNTLQPTEAAYIAGFLDGDGSIYAKLIPQPDYKDIKYGVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 44 - protein, 173 aa, artificial sequence
  I-MsoI variant
MATTKNTLQPTEAAYIAGFLDGDGSIYAKLIPAPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 45 - protein, 173 aa, artificial sequence
  I-MsoI variant
MATTKNTLQPTEAAYIAGFLDGDGSIYAKLIPRPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 46 - protein, 173 aa, artificial sequence
  I-MsoI variant
MATTKNTLQPTEAAYIAGFLDGDGSIYAKLIPHPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 47 - protein, 173 aa, artificial sequence
  I-MsoI variant
MATTKNTLQPTEAAYIAGFLDGDGSIYAKLIPDPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 48 - protein, 173 aa, artificial sequence
  I-MsoI variant
MATTKNTLQPTEAAYIAGFLDGDGSIYAKLIPDPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 49 - protein, 173 aa, artificial sequence
  I-MsoI variant
MATTKNTLQPTEAAYIAGFLDGDGSIYAKLIPQPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 50 - protein, 173 aa, artificial sequence
  I-MsoI variant
MATTKNTLQPTEAAYIAGFLDGDGSIYAKLIPPPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 51 - protein, 173 aa, artificial sequence
  I-MsoI variant
MATTKNTLQPTEAAYIAGFLDGDGSIYAKLIPKPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 52 - protein, 173 aa, artificial sequence
  I-MsoI variant
MATTKNTLQPTEAAYIAGFLDGDGSIYAKLIPKPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 53 - protein, 173 aa, artificial sequence
  I-MsoI variant
MATTKNTLQPTEAAYIAGFLDGDGSIYAKLIPKPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 54 - protein, 173 aa, artificial sequence
  I-MsoI variant
MATTKNTLQPTEAAYIAGFLDGDGSIYAKLIPKPDYKDIKYGMSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 55 - protein, 173 aa, artificial sequence
  I-MsoI variant
MATTKNTLQPTEAAYIAGFLDGDGSIYAKLIPSPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 56 - protein, 173 aa, artificial sequence
  I-MsoI variant
MATTKNTLQPTEAAYIAGFLDGDGSIYAKLIPHPDYKDIKYGVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 57 - protein, 173 aa, artificial sequence
  I-MsoI variant
MATTKNTLQPTEAAYIAGFLDGDGSIYAKLIPHPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 58 - protein, 173 aa, artificial sequence
  I-MsoI variant
MATTKNTLQPTEAAYIAGFLDGDGSIYAKLIPQPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 59 - protein, 173 aa, artificial sequence
  I-MsoI variant
MATTKNTLQPTEAAYIAGFLDGDGSIYAKLIPSPDYKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 60 - protein, 173 aa, artificial sequence
  I-MsoI variant
MATTKNTLQPTEAAYIAGFLDGDGSIYAKLIPAPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 61 - protein, 173 aa, artificial sequence
  I-MsoI variant
MATTKNTLQPTEAAYIAGFLDGDGSIYAKLIPHPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 62 - protein, 173 aa, artificial sequence
  I-MsoI variant
MATTKNTLQPTEAAYIAGFLDGDGSIYAKLIPCPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 63 - protein, 173 aa, artificial sequence
  I-MsoI variant
MATTKNTLQPTEAAYIAGFLDGDGSIYAKLIPHPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 64 - protein, 173 aa, artificial sequence
  I-MsoI variant
MATTKNTLQPTEAAYIAGFLDGDGSIYAKLIPCPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 65 - protein, 173 aa, artificial sequence
  I-MsoI variant
MATTKNTLQPTEAAYIAGFLDGDGSIYAKLIPSPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 66 - protein, 173 aa, artificial sequence
  I-MsoI variant
MATTKNTLQPTEAAYIAGFLDGDGSIYAKLIPRPDYKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 67 - protein, 173 aa, artificial sequence
  I-MsoI variant
MATTKNTLQPTEAAYIAGFLDGDGSIYAKLIPTPDYKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 68 - protein, 173 aa, artificial sequence
  I-MsoI variant
MATTKNTLQPTEAAYIAGFLDGDGSIYAKLIPAPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 69 - protein, 173 aa, artificial sequence
  I-MsoI variant
MATTKNTLQPTEAAYIAGFLDGDGSIYAKLIPGPDYKDIKYKVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 70 - protein, 173 aa, artificial sequence
  I-MsoI variant
MATTKNTLQPTEAAYIAGFLDGDGSIYAKLIPSPDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 71 - protein, 173 aa, artificial sequence
  I-MsoI variant
MATTKNTLQPTEAAYIAGFLDGDGSIYAKLIPHPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 72 - protein, 173 aa, artificial sequence
  I-MsoI variant
MATTKNTLQPTEAAYIAGFLDGDGSIYAKLIPQPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 73 - protein, 173 aa, artificial sequence
  I-MsoI variant
MATTKNTLQPTEAAYIAGFLDGDGSIYAKLIPQPDYKDIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 74 - protein, 173 aa, artificial sequence
  I-MsoI variant
MATTKNTLQPTEAAYIAGFLDGDGSIYAKLIPKPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 75 - protein, 173 aa, artificial sequence
  I-MsoI variant
MATTKNTLQPTEAAYIAGFLDGDGSIYAKLIPEPDYKDIKYWVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 76 - protein, 173 aa, artificial sequence
  I-MsoI variant
MATTKNTLQPTEAAYIAGFLDGDGSIYAKLIPKPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 77 - protein, 173 aa, artificial sequence
  I-MsoI variant
MATTKNTLQPTEAAYIAGFLDGDGSIYAKLIPNPDYKDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 78 - protein, 173 aa, artificial sequence
  I-MsoI variant
MATTKNTLQPTEAAYIAGFLDGDGSIYAKLIPHPDYKDIKYCVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 79 - protein, 173 aa, artificial sequence
  I-MsoI variant
MATTKNTLQPTEAAYIAGFLDGDGSIYAKLIPSPDYKDIKYAVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 80 - protein, 173 aa, artificial sequence
  I-MsoI variant
MATTKNTLQPTEAAYIAGFLDGDGSIYAKLIPRPDYKDIKYKVSLAISFIQRKDKIPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 81 - protein, 173 aa, artificial sequence
  I-MsoI variant
MATTKNALQPTEAAYIAGFLDGDGSIYAKLIPRPDYKDIKYKVSLAISFIQRKDKFPYLQDIYDQLGKRGNLRKDRGDGIADYTIIGSTHLSIMLPDLVPYLRIKKKQANRILHIINLYPQAQKNPSKFLDLVKIVDDVQNLNKRADELKSTNYDRLLEEFLKAGKIESSPAD

SEQ ID NO: 82 - protein, 173 aa, artificial sequence
  I-MsoI variant
MATTKNTLQPTEAAYIAGFLDGDGSIYAKLIPEPDYKDIKYTVSLAISFIQRKDKFPYLQDIYDQLGKRGNLRKDRGDGIADYTIIGSTHLSIILPDLVPYLRIKKKQASRILHIINLYPQAQKNPSKFLDLVKIVDDVQNLNKRADELKSTNYDRLLEEFLKAGKIESSPAD

SEQ ID NO: 83 - protein, 173 aa, artificial sequence
  I-MsoI variant
MATTKNTLQPTEAAYIAGFLDGDGSIYAKLIPGPDYKDIKYWVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 84 - protein, 173 aa, artificial sequence
  I-MsoI variant
MATTMNTLQPTEAAYIAGFLDGDGSIYAKLIPTPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 85 - protein, 173 aa, artificial sequence
  I-MsoI variant
MATTKNTLQPTEAAYIAGFLDGDGSIYAKLIPRPDSKNIKYQVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 86 - protein, 173 aa, artificial sequence
  I-MsoI variant
MATTKNTLQPTEAAYIAGFLDGDGSIYAKLIPHPDYKDIKYAVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 87 - protein, 173 aa, artificial sequence
  I-MsoI variant
MATTKNTLQPTEAAYIAGFLDGDGSIYAKLIPRPDYKDIRYSVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 88 - protein, 173 aa, artificial sequence
  I-MsoI variant
MATTKNTSQPTEAAYIAGFLDGDGSIYAKLIPKPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 89 - protein, 173 aa, artificial sequence
  I-MsoI variant
MATTKNTLQPTEAAYIAGFLDGDGSIYAKLIPRPDYNDIKYNVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 90 - protein, 173 aa, artificial sequence
  I-MsoI variant
MATTKNTLQPTEAAYIAGFLDGDGSIYAKLIPRLDYKDIKYPVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 91 - protein, 173 aa, artificial sequence
  I-MsoI variant
MATTKNTLQPTEAAYIAGFLDGDGSIYAKLIPTPDYKDIKYRVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSANYDRLLEEFLKAGKIESSPAD

SEQ ID NO: 92 - protein, 173 aa, artificial sequence
  I-MsoI variant
MATTKNTLQPTEAAYIAGFLDGDGSIYAKLIPRPDYKDIKYYVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKVGKIESSPAD

SEQ ID NO: 93 - protein, 173 aa, artificial sequence
  I-MsoI variant
MATTKNTLQPTEAAYIAGFLDGDGSIYAKLIPSPDYKDIKYHVSLAISFIQRKDKFPYLQDIYDQLGKRGNLRKDRGDGIADYTIIGSTHLSIILPDLVPYLRIKKKQANRILHIINLYPQAQKNPSKFLDLVKVVDDVQNLNKRADELKSTNYDRLLEEFLKAGKIESSPAD

SEQ ID NO: 94 - protein, 173 aa, artificial sequence
  I-MsoI variant
MATTKNTLQPTEAAYIAGFLDGDGSIYAKLIPRPDYKDIKYTVSLAISFIQRKDKFPYLQDIYDQLGKRGNLRKDRGDGIADHTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 95 - protein, 173 aa, artificial sequence
  I-MsoI variant
MATTKNTLQPTEAAYIAGFLDGDGSIYAKLIPHPDYKNIKYTVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 96 - protein, 173 aa, artificial sequence
  I-MsoI variant
MATTKNTLQPTEAAYIAGFLDGDGSIYAKLIPRPDYKDIKYNVNLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 97 - protein, 173 aa, artificial sequence
  I-MsoI variant
MATTKNTLQPTEAAYIAGFLDGDGSIYAKLIPKPDYKDIKYSVSLAISFIQRKDKFPYLQDIYDQLGKRGNLRKDRGDGIADYTIIGSTHLSIILPDLVPYLRIKKKQANRILHIINLYPQAQKNPSKFLDLVKMVDDVQNLNKRADELKSTNYDRLLEEFLKAGKIESSPAD

SEQ ID NO: 98 - protein, 173 aa, artificial sequence
  I-MsoI variant
MATTKNTLQPTEAAYIAGFLDGDGSIYAKLIPAPDYKDIKYKVSLAISFIQRKDKVPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 99 - protein, 173 aa, artificial sequence
  I-MsoI variant
MATTKNTLQPTEAAYIAGFLDGDGSIYAKLIPRPDYKDIKYSVSLAISYIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 100 - DNA, 50 bp, artificial sequence
  target oligo
tggcatacaagtttcnnnacgtcgtacgacgtnnngacaatcgtctgtca

SEQ ID NO: 101 - DNA, 24 bp, artificial sequence
  Gal10R primer
acaaccttgattggagacttgacc

SEQ ID NO: 102 - DNA, 38 bp, artificial sequence
  MlibF1 primer
ctagcaatttcttttatacaaagaaaagataaatttcc

SEQ ID NO: 103 - DNA, 26 bp, artificial sequence
  Gal10F primer
gcaactttagtgctgacacatacagg

SEQ ID NO: 104 - DNA, 22 bp, artificial sequence
  10 GGG_P target
cgggacgtcgtacgacgtcccg

SEQ ID NO: 105 - protein, 173 aa, artificial sequence
  I-MsoI scaffold protein
MATTKNTLQPTEAAYIAGFLDGDGSIYAKLIPRPDYKDIKYQVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSPAD

SEQ ID NO: 106 - DNA, 60 bp, artificial sequence
  Mlib1R primer
aaaagaaattgctagactcacmbnatatttaatgtctttgtaatcaggmbnaggaataag